(19) United States
(12) Patent Application Publication
(10) Pub. No.: US 2007/0105144 A1
(43) Pub. Date: May 10, 2007

(54) Method of nucleic acid analysis

(75) Inventors: Kohshi Maeda, Hitachinaka (JP); Shinichi Fukuzono, Hitachinaka (JP)

Correspondence Address: MCDERMOTT WILL & EMERY LLP, 600 13TH STREET, N.W., WASHINGTON, DC, 20005-3096, US

(73) Assignee: HITACHI HIGH-TECHNOLOGIES CORPORATION

(21) Appl. No.: 11/593,606

(22) Filed: Nov. 7, 2006

(30) Foreign Application Priority Data
Nov. 7, 2005 (JP) 2005-322224

(51) Int. Cl. C12Q 1/68; C07H 21/04

(52) U.S. Cl. 435/6; 536/25.32

(57) ABSTRACT

It is intended to provide a method of efficient, high-precision quantitative analysis of a gene region with plural possible sequences by simultaneously separating and detecting standard and measurement nucleic acids overlapping in their detection times. The present invention provides a method of quantitative analysis of a gene region with plural possible sequences, comprising separately labeling a standard nucleic acid sample and a measurement nucleic acid sample with substances of different molecular weights and simultaneously separating and detecting nucleic acid fragments in the gene regions derived from the standard nucleic acid sample and from the measurement nucleic acid sample, thereby quantitatively analyzing the nucleic acid fragments.

### CLAIM OF PRIORITY

The present application claims priority from Japanese application JP 2005-322224 filed on Nov. 11, 2005, the content of which is hereby incorporated by reference into this application.

### BACKGROUND OF THE INVENTION

1. Field of the Invention

The present invention relates to a method of high-precision quantitative analysis of a nucleic acid fragment in a gene region with plural possible sequences.

2. Background Art

In separation and detection by nucleic acid electrophoresis, since nucleic acid fragments overlapping in their detection times are detected at the same time, a standard nucleic acid and a measurement nucleic acid must be separated and detected separately, followed by the correction of the difference between their migrations and the subsequent comparative analysis of migration waveforms. Therefore, the analysis of the nucleic acid fragments overlapping in their detection times presented the following problems in terms of the labor, time, and cost of the analysis in conventional methods of nucleic acid analysis:

since the nucleic acid fragments of identical gene regions in the standard and measurement nucleic acids are separately analyzed, at least two runs of analysis are necessary for one gene region, and the number of an analysis run is increased with increase in the number of the measurement nucleic acid, resulting in poor analysis efficiency; and

the correction of detection time difference in nucleic acid electrophoresis requires an internal control for detection time correction and therefore requires cost for producing the internal control, the step of adding it, and labor for correcting the difference between the migrations.

A possible means for solving these problems is a method of simultaneous detection by one run of nucleic acid electrophoresis comprising subjecting identical gene regions in standard and measurement nucleic acids to nucleic acid amplification using primers that are complementary to the gene regions and are labeled with fluorescent dyes differing between the standard and measurement nucleic acids, mixing their respective amplification products, and simultaneously detecting them (JP Patent Publication (Kokai) No. 5-322771A (1993), JP Patent Publication (Kokai) No. 2000-35432A, JP Patent Publication (Kokai) No. 6-125797A (1994)). However, simultaneous detection with high quantitative properties found difficulty for reasons described below.

First, the nucleic acid fragments of the identical gene regions in the standard and measurement nucleic acids are detected with them overlapping by using the method described above. This holds true of the case where two or more nucleic acid fragments with the same detection times are labeled with fluorophores having different fluorescence spectra. The conventional methods allows for the separate quantification of the standard and measurement nucleic acids by correcting the overlap with inverse matrix on the bases of previously recorded fluorescence spectrum data corresponding to each fluorophore used for the labeling and thereby detecting the standard and measurement nucleic acids by the different fluorescent dyes. However, the fluorescence spectrum data used in this correction have been determined relative to standard fluorescent substances. Therefore, errors are made in correction results unless the fluorescence spectra of the fluorophores actually used in the measurement completely agree with the data. In fact, the fluorescence spectra of the fluorophores fluctuate depending on storage conditions thereof and so on and therefore rarely completely agree with the data. Thus, when two different fluorescent dyes overlap each other, quantitative detection is difficult. For example, when the intensities of two waveforms of fluorescent labels having different fluorescence wavelengths but the same detection times differ by 10 times or more, the phenomenon where the smaller waveform disappears due to correction is confirmed. Therefore, quantitative properties equal to conventional method could not be maintained, and analysis such as nucleic acid level comparison, which requires quantitative properties, was difficult.

An object of the present invention is to provide a method of efficient, high-precision quantitative analysis of a gene region with plural possible sequences by simultaneously separating and detecting standard and measurement nucleic acids overlapping in their detection times.

### SUMMARY OF THE INVENTION

As a result of diligent studies for the object, it has been found that standard nucleic acid- and measurement nucleic acid-derived nucleic acid fragments amplified even from identical gene regions can be allowed to differ in detection time from each other in nucleic acid electrophoresis depending on the combination of different fluorescent dyes with which the standard and measurement nucleic acids have been labeled separately by use of primers complementary to the target gene regions. It has also been found that when the nucleic acid fragments amplified from the identical gene regions in the standard and measurement nucleic acids are simultaneously detected by use of this difference in detection time under conditions that prevent detection waveforms from overlapping, the target gene regions may be analyzed quantitatively at the same time.

Namely, the object of the present invention can be attained by the following means:

1) identical gene regions in standard and measurement nucleic acids are separately amplified with primers labeled with fluorescent dyes that permit the detection times of nucleic acid fragments of the gene regions to be detected to differ between the standard and measurement nucleic acids;

2) the nucleic acid fragments amplified from the standard and measurement nucleic acids are mixed;

3) the mixed nucleic acid fragments are separated and detected by nucleic acid electrophoresis;

4) the difference between detection times derived from the fluorescent labels is corrected; and

5) waveforms are comparatively analyzed.

Based on these findings, the present invention provides a method of quantitative analysis of a gene region with plural possible sequences: comprising separately labeling a standard nucleic acid sample and a measurement nucleic acid sample with substances of different molecular weights; simultaneously separating and detecting nucleic acid fragments in the gene regions derived from the standard nucleic acid sample and from the measurement nucleic acid sample; and thereby quantitatively analyzing the gene region with plural possible sequences.

The method may further comprise the step of correcting the difference between the detection times of the nucleic acid fragments by image processing.

Nucleic acid electrophoresis can be utilized as a means for separating and detecting nucleic acid fragments. In a certain aspect, the nucleic acid fragments are separated and detected by a single strand conformation polymorphism (SSCP) method utilizing nucleic acid electrophoresis.

The labeling of the standard nucleic acid sample and the measurement nucleic acid sample is performed by separate nucleic acid amplification using primers comprising the substances of different molecular weights.

Fluorescent substances, nucleic acid-binding proteins, and inorganic substances such as radioactive substances can be used as the labeling substances. In the present invention, the fluorescent substances are particularly preferable.

Examples of the combination of the labeling substances of different molecular weights can include FAM™ and ROX™, FAM™ and TAMRA™, fluorescein isothiocyanate (FITC) and Texas Red, FITC and phycoerythrin (PE), and the combination of polynucleotides of different molecular weights such as polyA and polyT.

The method of nucleic acid analysis of the present invention can be applied to the quantitative analysis of the gene region with plural possible sequences such as polymorphism and mutation sites and is particularly useful in loss of heterozygosity (LOH) analysis and cancer cell detection.

According to the present invention, nucleic acid fragments in the same gene regions of different origins can be simultaneously detected in a single lane or a single capillary at electrophoresis.

### BRIEF DESCRIPTION OF THE DRAWINGS

FIG. 1 shows a schematic view of an analysis method according to the present invention;

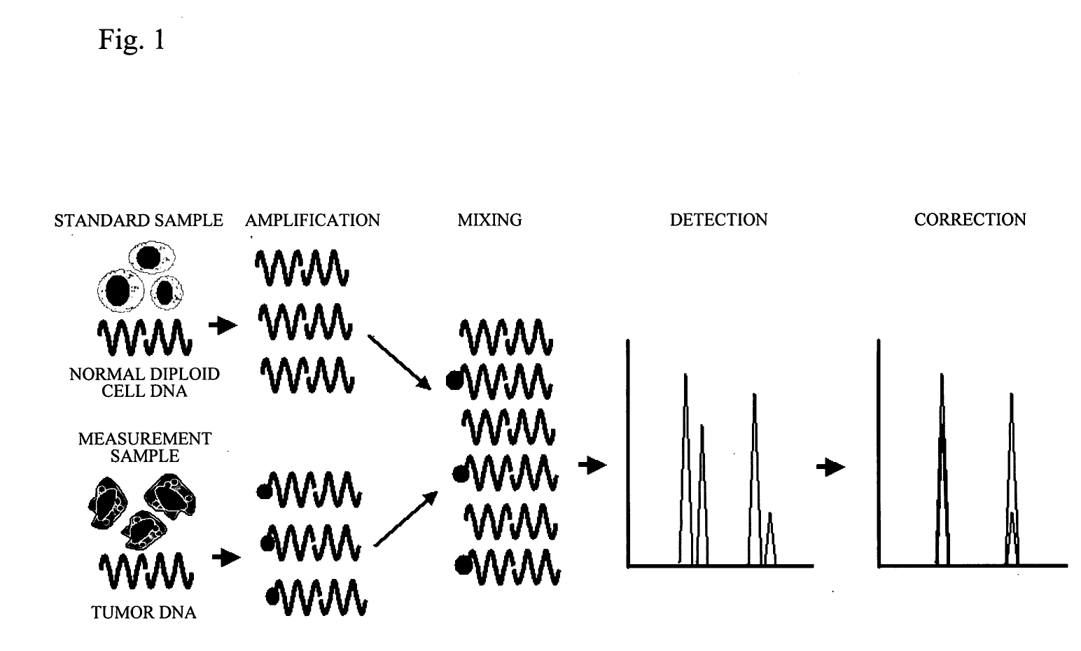

FIG. 2 shows a comparative diagram of before- and after-correction in fluorescence multiplexing analysis using a SSCP method;

FIG. 3 shows a comparative diagram of before- and after-correction with an internal control for position correction in individual analysis using the SSCP method;

FIG. 4 shows detection waveforms and a correction diagram of detection time difference in gene mutation analysis using the present invention; and

FIG. 5 shows detection waveforms and a correction diagram of detection time difference in LOH analysis using the present invention.

### DETAILED DESCRIPTION OF THE PREFERRED EMBODIMENTS

Hereinafter, the above-described and additional novel characteristics and advantages of the present invention will be described with reference to drawings.

The present invention provides a quantitative assay of nucleic acid samples capable of reducing the number of an analysis run in electrophoresis and simplifying analysis steps by simultaneously detecting, in the analysis of identical gene regions in standard and measurement nucleic acids, amplified fragments of the identical gene regions contained in the standard and measurement nucleic acids, with high quantitative properties maintained.

A “sample” intended by the present invention refers to all nucleic acid-containing biological samples including, but not particularly limited to, samples in examinations such as mass screening, medical check-up, dock examination, and posting examination, the blood, tissue, and urine of outpatients and inpatients in hospitals, and animals, plants, and microorganisms.

A “nucleic acid” intended by the present invention refers to all nucleic acids including DNA and RNA extracted from the sample. To extract the nucleic acid, a method known in the art can be used.

In the present invention, a “measurement nucleic acid (sample)” refers to a nucleic acid (sample) to be measured, and a “standard nucleic acid (sample)” refers to a nucleic acid (sample) used for comparison with the measurement nucleic acid (sample) and is preferably a nucleic acid (sample) of a gene region identical to that of the measurement nucleic acid, more preferably a nucleic acid (sample) of the identical gene region derived from the same individual. Specifically, a nucleic acid of a particular gene region extracted from the colon cancer tissue of a patient with colon cancer is referred to as the measurement nucleic acid, whereas a nucleic acid of an identical gene region extracted from a leukocyte separated from the peripheral blood of the same patient is referred to as the standard nucleic acid.

An apparatus for analyzing nucleic acid used in the present invention is not particularly limited as long as it can quantitatively separate and detect plural nucleic acid fragments with high sensitivity according to difference in molecular size or conformation. Concrete examples thereof include gel electrophoresis, capillary electrophoresis, and chip electrophoresis apparatuses, more concretely Sequi-Gen GT Sequencing Cell (Bio-Rad) and Genetic Analyzer (Applied Biosystems Inc.) used in nucleic acid sequencing and fragment analysis, Capillary Electrophoresis System (Agilent Technologies Inc.) that can analyze various substances such as metal ions, nucleic acids, and proteins by electrophoresis, and Cosmo-i (Hitachi High-Technologies Corporation) and Agilent 2100 Bio Analyzer (Agilent Technologies Inc.) using a microchannel chip. Although the capillary electrophoresis apparatus is particularly preferable, the present invention is not limited to the analyzing apparatuses described above and can employ any apparatus that can separate and detect nucleic acid fragments with high separation ability.

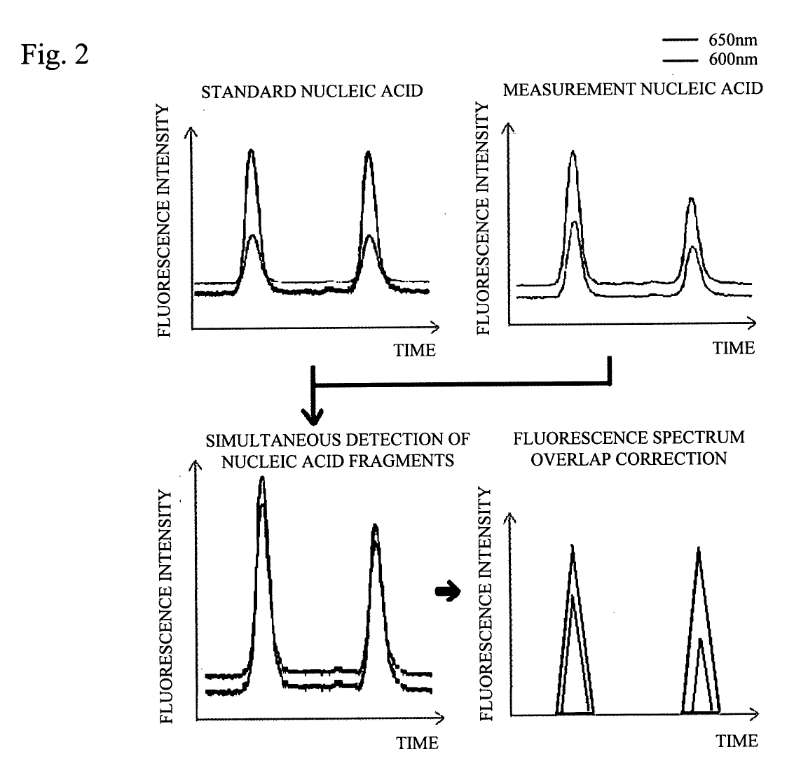

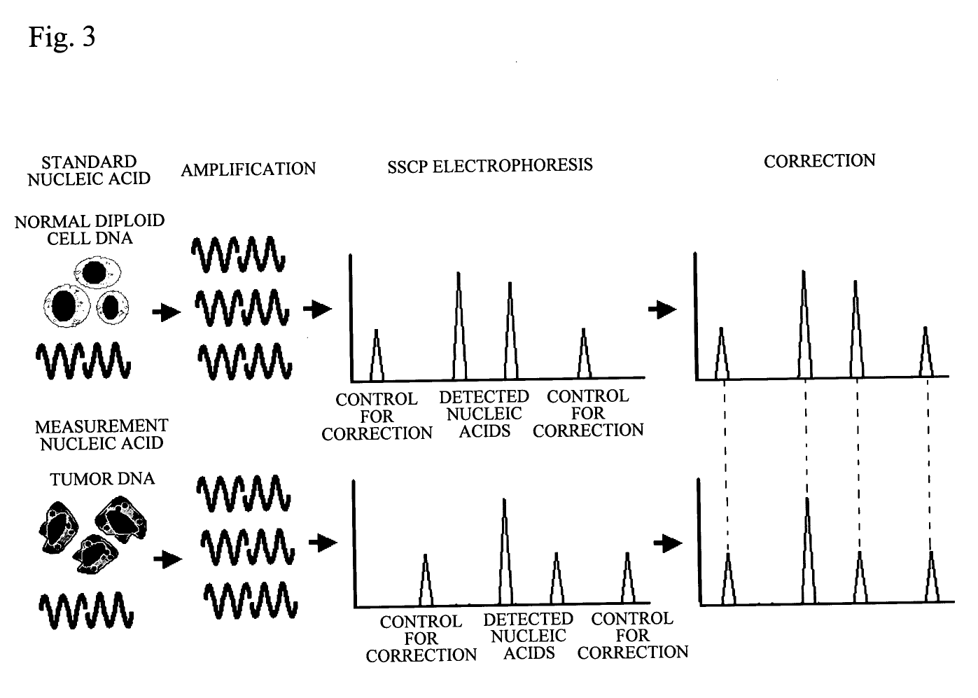

In conventional methods, each of these apparatuses presents its own problems. For example, the apparatus using a gel plate has a throughput problem attributed to long electrophoresis time and much labor required for producing the gel plate. Moreover, when amplified fragments derived from standard and measurement nucleic acids composed of identical sequences are compared, it is sometimes impossible to add nucleic acid fragments overlapping in their detection times or the standard and measurement nucleic acids to the same lane or capillary because some capillary electrophoresis and chip electrophoresis apparatuses are not designed to detect plural fluorescent dyes. As described in FIG. 3, when several electrophoresis apparatuses including capillary electrophoresis and chip electrophoresis apparatuses are used to electrophorese amplified nucleic acid fragments with the same detection times in different lanes or capillaries, a detection time lag occurs between the lanes or capillaries due to the individual channels. Therefore, the comparison of detection peaks such as detection time between the different lanes or capillaries inevitably requires mixing an internal control for position correction and therefore has a problem leading to increased cost and labor. The Genetic Analyzer (Applied Biosystems Inc.) capable of using plural fluorescent labels allows for the detection of nucleic acids with totally the same detection times from the same lane or capillary by using different fluorescent labels and detecting a fluorescence wavelength optimum for each fluorescent label. However, when two different fluorescent dyes overlap each other as shown in FIG. 2, the fluorescence spectrum overlap cannot be corrected accurately. Therefore, this apparatus presents a problem in quantitative comparison based on their respective peak areas or intensity.

The method of nucleic acid analysis according to the present invention allows for quantitative peak analysis by simultaneously electrophoresing plural nucleic acid fragments overlapping in their detection times in the same lane or capillary, detecting two nucleic acid fragments, which originally have the same detection times, at different detection times, and correcting the detection times without the use of an internal control for detection time correction.

Specifically, the method of nucleic acid analysis according to the present invention comprises the steps of amplifying the gene region to be detected, mixing the amplified fragments of the standard and measurement nucleic acids, separating and detecting the amplified fragments, and identifying each amplified fragment on the basis of detection time difference derived from the labeling molecules.

In the present invention, a method for amplifying the gene region to be detected is not particularly limited and includes polymerase chain reaction (PCR), strand displacement analysis (SDA), isothermal and chimeric primer-initiated amplification of nucleic acid (ICAN), and loop-mediated isothermal amplification (LAMP). Particularly, for example, the method disclosed in JP Patent Publication (Kokai) No. 9-201199A (1997) can be utilized as a method of LOH detection by PCR using Taq-polymerase, wherein the ends of PCR products after amplification are treated and made into blunt-ends, and the resulting nucleic acid fragments are analyzed by SSCP.

In PCR, the gene region to be amplified is not particularly limited as long as it falls within a range that contains a region with plural possible sequences such as at least one gene polymorphism or mutation. A length and range easily amplified by PCR must be selected. Preferably, the gene region may be 100 to 200 bp in length and fall within a range determined in light of the design of oligonucleotide primers suitable for PCR.

In the present invention, the standard and measurement nucleic acids are labeled with substances of different molecular weights in order to cause their detection times to differ from each other. Alternatively, one or more bases are added to the 5′ ends of primers for the standard nucleic acid or the measurement nucleic acid or both. The labeling can be conducted by modifying the standard nucleic acid or the measurement nucleic acid or both with substances of different molecular weights, or by amplifying the standard nucleic acid or the measurement nucleic acid or both using primers comprising one or more additional bases at the 5′ end thereof.

The labeling substance can include fluorescent substances, nucleic acid-binding proteins, and inorganic substances such as radioactive substances. In the present invention, the fluorescent substances are particularly preferable. Examples of the combination of the labeling substances of different molecular weights can include FAM™ and ROX™, FAM™ and TAMRA™, fluorescein isothiocyanate (FITC) and Texas Red, FITC and phycoerythrin (PE), and the combination of polynucleotides of different molecular weights such as polyA and polyT. The bases added to the 5′ ends of the primers are not particularly limited by the type and number of added bases as long as the detection times of the standard and measurement nucleic acids differ from each other.

Analysis using the Genetic Analyzer (Applied Biosystems Inc.) allows for the simultaneous detection and separation of the standard and measurement nucleic acids when amplification primers are labeled with fluorescent dyes of molecular weights that differ from each other as much as possible. However, the present invention is not limited to the example described above as long as the standard and measurement nucleic acids can be detected at different detection times by labeling them with molecules that give detection time difference. It is more preferable that the amount of change in detection time by the labeling of the molecules should be confirmed in advance.

When PCR is used as the amplification method, a thermal cycler, for example, MJ Research serving as an apparatus for amplifying nucleic acid (MJ Research) is used as an apparatus for amplifying nucleic acid in the present invention. Such an apparatus for amplifying nucleic acid utilizing PCR amplifies nucleic acids by repeating heating and cooling for a microcontainer filled with a solution adjusted with reagents for nucleic acid amplification. When a nucleic acid amplification method performed at low temperatures is used, concrete examples of apparatuses for this purpose include thermo heaters and incubators. The present invention is not limited to these apparatuses and can employ any apparatus that is capable of nucleic acid amplification.

The amplified fragments derived from the standard and measurement nucleic acids can be mixed, for example, by aliquoting the amplified fragments amplified from the standard nucleic acid and the measurement fragments amplified from the measurement nucleic acid in equal amounts and mixing them with a vortex mixer.

In the present invention, any method may be used as a method for separating and detecting the nucleic acid fragments as long as it is capable of highly quantitative separation and detection of plural amplified nucleic acid fragments according to difference in molecular size or conformation. One of preferable methods can include a single strand conformation polymorphism (SSCP) method. The SSCP method is a method wherein each amplified fragment is denatured into single strands, which in turn form conformations, and the nucleic acid fragments are separated and detected on the basis of the difference between the conformations determined by difference by one or more bases between the gene sequences. This method can separate and detect nucleic acid fragments based on gene mutation, gene polymorphism (e.g., single nucleotide polymorphism, single nucleotide deletion mutation, and microsatellite polymorphism differing in repeat unit by one or more units), and the difference between the sizes of the labeling molecules on the nucleic acids.

Specifically, the method of the present invention is applicable to, but not limited to, a method for analyzing the viability of cells having mutated genes and a method for analyzing the phenomenon of loss of heterozygosity (LOH; at least partial deletion of either of chromosomes in a sample) by using gene polymorphism (hereinafter, referred to as the LOH analysis method).

In the present invention, the gene “mutation” means the phenomenon where change such as substitution or deletion is observed in a portion of the gene. For example, in human cancer, many mutations have been observed in gene regions such as transcriptional regulators, which are involved in genome replication, gene repair, and so on. More specifically, examples of the mutation in the present invention include, but not limited to, mutations in the gene region of a tumor suppressor gene p53 and in the gene region of an oncogene K-ras.

In the analysis of the viability of cells having these mutated genes, a gene region containing a gene that is easily mutated as illustrated above is amplified with amplification primers complementary thereto and detected with a test marker.

More specifically, the detection times of the amplified fragments obtained from the standard and measurement nucleic acids are compared, and the ratio between normal and mutated gene fragments of the measurement nucleic acid extracted from a mixture of cells where the gene region to be detected is normal and cells where the gene region to be detected is mutated is compared with respect to this ratio of the detection times. Namely, the measurement nucleic acid is analyzed for the ratio between the normal cells without mutation in the gene region to be detected and the cells with mutation in this gene region. Thus, in such gene mutation analysis, the ratio between the amplified fragments derived from normal and mutated genes in the measurement sample is the proportion of abnormal cells in the measurement sample. The highly sensitive analysis of this ratio requires high quantitative properties.

In the present invention, the “gene polymorphism” refers to genetic polymorphism on chromosomes and is derived from individual differences of nucleotide sequences on genes. Single nucleotide polymorphism has been said to exist with a frequency of approximately one base out of several hundreds of bases on average. For example, gene polymorphism by one-nucleotide substitution in the intron 8 of a human aldolase B gene (ALDOB) on chromosome 9 was identified on 9q22 and reported by Brooks et al (Am. J. Hum. Genet. 52, p. 835-840, Brooks et al., (1993)). Alternatively, gene polymorphism by T/C substitution on a VAV2 gene identified on 9q34 is founded on the measurement data of the nucleotide sequence of a cosmid clone L196C8 strain (Gene Bank Registration No. AC002111) and was found by National Cancer Center Chuo Hospital.

In the present invention, the “microsatellite polymorphism site differing in repeat unit by one or more units” refers to a site where provided that a repeat unit is composed of, for example, 4 particular base pairs, this unit of base pairs is repeated many times in a certain region on a gene, and the number of the repeat differs among alleles and thereby forms gene polymorphism. Since the difference in repeat among alleles makes peak separation sharp, resulting in improved analysis precision, such microsatellite polymorphism can be analyzed by the repeat unit difference by one or more units. However, in the microsatellite polymorphism, PCR amplification efficiency varies among alleles depending on difference in repeat length in heterozygotes, and the ratio of the amounts of alleles sometimes differs among blood samples from a patient used as the standard nucleic acid. There are some other problems such that the microsatellite polymorphism is inconvenient for automation to automatically detect alleles because the positions of occurrence of the alleles can not be defined in SSCP due to the difference in the length of the repeat sequence. For example, D9S775, D9S304, and D9S303 are known as microsatellite polymorphisms on chromosome 9, and the information thereof can be obtained from the genome database (Internet address: http://gdbwww.gdb.prg/).

Alleles in paternally derived and maternally derived chromosomes can be distinguished from each other by analyzing such gene polymorphism. Namely, in a heterozygote (individual with paternal and maternal alleles differing from each other) for a certain gene polymorphism site, deletion in its gene itself can be detected by amplifying both the paternal and maternal alleles, comparing the amplification levels of these alleles, and measuring at least partial deletion of either a paternal or maternal chromosome (LOH). More specifically, the ratio of the amplification levels of paternal and maternal alleles of the measurement nucleic acid extracted from a mixture of cells with normal chromosomes and cells with deletions in the chromosomes is compared with respect to the ratio of the amplification levels of paternal and maternal alleles obtained from the standard nucleic acid. Namely, the measurement nucleic acid is analyzed for the ratio between the normal cells without deletions in the chromosomes and the abnormal cells with deletions in the chromosomes. Thus, in the LOH analysis, the difference between the amplification levels of the paternally derived and maternally derived alleles is the proportion of the cells with abnormal chromosomes. The highly sensitive analysis of this ratio requires high quantitative properties.

In the present invention, the “at least partial deletion of either of chromosomes” includes the complete deletion or partial deletion of either of autosomes (homologous chromosomes) and the partial deletion of gene sites differing between the homologous chromosomes.

When single nucleotide polymorphism (SNP) is utilized, heterozygote frequency is 50% at a maximum. Therefore, the examination of deletion in one chromosome requires combining several gene polymorphisms with high heterozygote occurrence frequency including microsatellite polymorphism, in addition to the single nucleotide polymorphism. By such combination of several gene polymorphisms, the judgment rate of match of individuals from which the standard and measurement nucleic acids are derived is improved. Thus, the match of individuals from which the nucleic acids are derived may also be confirmed in parallel with the analysis of LOH, gene mutation, or the like.

To identify each amplified fragment on the basis of detection time difference derived from the labeling molecules, the difference between the detection time of each nucleic acid and the detection time of the nucleic acid labeled with one of the molecules of different sizes is examined in advance, and each allele peak is determined based on the detection times. It is more preferable that the peak should be allowed to move in parallel by image processing to correct the detection time difference derived from the labeling molecules, thereby confirming the agreement between the peaks derived from the standard and measurement nucleic acids.

In this way, the homo- or hetero-type of each polymorphism or the number of repeat units in microsatellite polymorphism is determined from the positions of occurrence of peaks of plural gene regions containing gene polymorphism such as single nucleotide polymorphism and microsatellite polymorphism.

In the present invention, the “image processing” refers to a method for correcting waveform images or waveform data from electrophoresis for the difference between detection times derived from the labeling molecules. Specifically, the image processing can be done by indicating detection peaks derived from the standard and measurement nucleic acids with them overlaid and indicating each peak by its own distinct color or line, or otherwise, by determining the detection peaks derived from the standard and measurement nucleic acids on the basis of their detection times and indicating each peak by its own distinct color or line.

### EXAMPLES

Hereinafter, the present invention will be described more fully with reference to Examples. However, the present invention is not intended to be limited to these Examples.

### Example 1

### Measurement of Cancer Cell Ratio by LOH Analysis

1. Acquisition of Samples

Patient samples from which standard and measurement nucleic acids were derived were obtained, then treated, and stored until LOH analysis, as described below. Peripheral blood leukocytes (PBL) were used for the standard nucleic acid. Into a blood collecting device containing heparin, 10 mL of venous blood was collected and spun down at 3,000 rpm for 15 minutes. Then, the plasma was discarded. The resulting blood sample was hemolyzed by adding 40 mL of 0.2% saline to this blood collecting device, and spun down again at 3,000 rpm for 15 minutes, and the supernatant fluid was then discarded. Similar procedures were repeated twice. The remaining pellet was cryopreserved at −80° C. For the measurement nucleic acid, a portion of fresh tissues obtained by resection or biopsy was put into a 1.5-ml tube and cryopreserved at −80° C.

2. Nucleic Acid Extraction

Human genomic nucleic acids from the blood and the tissue were extracted according to the method of Davis et al (Basic Methods in Molecular Biology, published by Elsevier Science Publishing) or Sugano et al (Lab. Invest. 68, p. 361-366, Sugano et al., (1993)) involving protease K digestion and subsequent extraction with phenol/chloroform. In brief, after treatment at 65° C. for 15 minutes, each sample was supplemented with 10 mM Tris-HCl buffer containing 1 mg/mL protease K, 10 mM ethylenediamine tetraacetic acid (EDTA), and 150 mM NaCl and incubated overnight at 37° C. To this reaction solution, an equal amount of phenol:chloroform (1:1) solution was added and the mixture centrifuged to extract a nucleic acid. To this extract, 0.1 volumes of 3 mol/L sodium acetate solution and 2.5 volumes of cold dehydrated ethanol were added and cooled at −20° C. for 2 hours to precipitate the nucleic acid. To the urine and cancer tissue samples, 1 μg of glycogen was added as a carrier for ethanol precipitation to improve the rate of nucleic acid collection. This solution was spun down to collect a precipitate, which was further washed by adding 1 mL of 80% ethanol thereto and dried with a vacuum centrifugal concentrator. This nucleic acid-containing precipitate was remelted with a TE buffer solution and cryopreserved at −25° C.

3. Amplification of Gene Regions to be Detected

PCR amplification conditions used in this Example were summarized in Table 1.

TABLE 1
PCR amplification conditions

| | Temp (° C.) | Time (min) |
|---|---|---|
| 1 | 95 | 5:00 |
| 2 | 95 | 0:30 |
| 3 | 57 | 0:30 |
| 4 | 72 | 0:30 |
| 5 | 72 | 7:00 |
| 6 | 4 | for ever |

Step2-Step4: 30 cycle

Either a forward or reverse PCR primer for standard nucleic acid amplification was labeled at the 5′ end thereof with a FAM™ fluorescent dye (molecular weight: 473.4, 495 nm for excitation/519 nm for fluorescence), while either a forward or reverse PCR primer for measurement nucleic acid amplification was labeled at the 5′ end thereof with a ROX™ fluorescent dye (molecular weight: 668.15, 576 nm for excitation/601 nm for fluorescence). p53 and K-ras gene regions were used as gene regions from which gene mutations were detected.

TABLE 2

| | | | | |
|---|---|---|---|---|
| Gene | | Direc- | | |
| name | Label | tion | 5′-Sequence-3′ | SEQ ID NO |

Primers for standard nucleic acid amplification

| | | | | |
|---|---|---|---|---|
| p53 | FAM | Forward | TCC ATC TAC AAG | SEQ ID NO: 1 |
| | | | CAG TCA | |
| | None | Reverse | CAG ACC TAA GAG | SEQ ID NO: 2 |
| | | | CAA TCA | |
| K-ras | FA, M | Forward | CCT GCT GAA AAT | SEQ ID NO: 3 |
| | | | GAC TGA A | |
| | None | Reverse | CAT GAA AAT GGT | SEQ ID NO: 4 |
| | | | CAG AGA AA | |

Primers for measurement nucleic acid
amplification

| | | | | |
|---|---|---|---|---|
| p53 | ROX | Forward | GGG CTT GAC TTT | SEQ ID NO: 5 |
| | | | CCA ACA CG | |
| | None | Reverse | TCT AGC CTC AAT | SEQ ID NO: 6 |
| | | | CCT CAT AC | |
| K-ras | ROX | Forward | GTG TCT GCA CTG | SEQ ID NO: 7 |
| | | | GCC ACA CT | |
| | None | Reverse | TCC AAA GGA CCT | SEQ ID NO: 8 |
| | | | TCT CCA AA | |

The total amount of a solution was brought to 30 μl by adding 0.1 μg of the genomic nucleic acid (template) extracted from each biological sample, 1.0 pM each primer, 10 nM each deoxyribonucleotide triphosphate (dNTP), 10 μM Tris-HCl buffer (pH 8.3), 50 mM KCl, 1.5 mM MgCl₂, 0.001% (w/v) gelatin, and 0.75 units of Taq DNA polymerase (Perkin Elmer). This solution was subjected to PCR reaction under the conditions shown in Table 1. After PCR reaction, the resulting solution was stored on ice (4° C.), and 5 μL of the PCR reaction solution amplified from the standard nucleic acid and 5 μL of the PCR reaction solution amplified from the measurement nucleic acid were mixed with a vortex mixer to prepare a mixed PCR reaction solution.

4. Blunt-End Treatment In blunt-end treatment, reagents and the samples were added in the order shown below.

However, the adding order is not limited to this example. The mixed PCR reaction solution was treated with a Klenow fragment (manufactured by TAKARA) to make the 3′ end thereof into a blunt-end. The Klenow fragment added at 0.5 units was reacted with 5 μL of the mixed PCR reaction solution at 37° C. for 30 minutes (Genes, Chromosomes & Cancer, 15, p. 157-164, Sugano et al., (1996)). After reaction, 1 μL of 100 mM EDTA was added thereto to perform enzyme inactivation treatment.

5. Preparation of Samples for Analysis

In the preparation of samples for analysis, reagents and the blunt-end-treated samples were added in the order and volumes shown below. However, the adding order, volumes, and denaturation conditions are not limited to this example as long as the nucleic acid fragments are denatured. Specifically, 1.0 μL of undiluted solution of the DNA amplification products was added to 39 μL of DNA denaturant formamide placed in an analytical microcontainer to adjust the total volume to 40 μL, then denatured with heat at 92° C. for 2 minutes, and rapidly cooled on ice (4° C.) for 5 minutes.

6. SSCP Electrophoresis

The samples prepared for analysis were subjected to SSCP electrophoresis with Tris-HCl and glycine as an electrophoresis buffer and 15% GeneScan Polymer as a separation polymer under conditions of sample introduction: at a voltage of 20 kV for 5 seconds and electrophoresis: at a voltage of 15 kV for 70 minutes.

7. Detection Time Correction

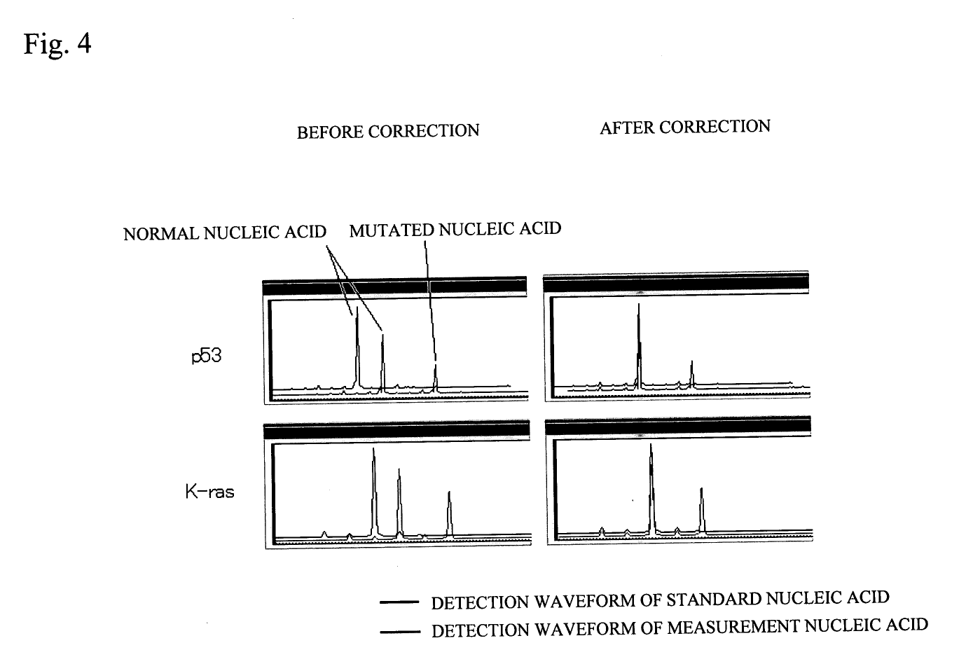

In gene mutation detection using a SSCP method, it is difficult to differentiate between peaks derived from a normal nucleic acid and from a gene-mutated nucleic acid due to an unspecified majority of gene mutation sites only by using the peak of the measurement nucleic acid. Thus, in this Example, the ratio of a mutated nucleic acid in the measurement sample was analyzed by simultaneously detecting the standard and measurement nucleic acids in the same capillary and thereby determining a peak derived from the normal nucleic acid. The standard nucleic acid labeled with the FAM fluorescent dye was detected with the peak of the standard nucleic acid amplified fragment having a fluorescence wavelength around 520 nm, while the measurement nucleic acid labeled with the ROX fluorescent dye was detected with the peak of the measurement nucleic acid amplified fragment having a fluorescence wavelength around 600 nm. In this detection, the ROX-labeled measurement nucleic acid had a molecular weight larger than that of the FAM-labeled standard nucleic acid and therefore resulted in a detection time approximately 4 seconds later, as shown in the left column of FIG. 4. Thus, the peak was allowed to move in parallel by image processing to correct the detection time difference by 4 seconds, as shown in the right column of FIG. 4. Then, the agreement of the standard nucleic acid-derived peak (NA0) with the peak derived from the normal nucleic acid (TA0) in the measurement nucleic acid was confirmed in each gene region to determine the normal nucleic acid-derived peak.

8. Measurement of Ratio of Cell with Gene Mutation

To measure the ratio of a cell with gene mutation contained in the measurement sample, the ratio of a mutated nucleic acid A1 in a measurement sample T containing a normal nucleic acid A0 may be determined. Namely, the ratio can be measured according to the method represented by the following formula:
Cancer cell ratio in measurement sample (%)=TA0×100/(TA0+TA1).

In the formula, TA0 and TA1 denote the peak intensity of each signal. A cancer cell ratio (%) in the measurement sample was measured by using the calculation formula for calculating a mutated cell ratio (Table 3).

TABLE 3
Measurement result of ratio of cell with gene mutation

| Gene name | p53 | K-ras |
|---|---|---|
| Mutated cell ratio (%) | 32% | 43% |

As described above, the standard and measurement nucleic acids were detected in the same capillary to prevent their peaks from overlapping. Then, the detection times were corrected after detection. As a result, fluorescence intensity overlap was prevented, and normal nucleic acid- and mutated nucleic acid-derived peaks were determined to allow for quantitative analysis.

### Example 2

### Measurement of Ratio of Cell with Gene Mutation by Mutation Analysis

1. Acquisition of Samples

Samples were obtained and stored in the same way as in Example 1.

2. Nucleic Acid Extraction

Nucleic acids were extracted and stored in the same way as in Example 1.

3. Amplification of Gene Regions to be Detected

Amplification was performed in the same way as in Example 1 using the same gene amplification reaction conditions and reagent composition. Either a forward or reverse PCR primer for standard nucleic acid amplification was labeled at the 5′ end thereof with a FAM™ fluorescent dye (molecular weight: 473.4, 495 nm for excitation/519 nm for fluorescence), while either a forward or reverse PCR primer for measurement nucleic acid amplification was labeled at the 5′ end thereof with a ROX™ fluorescent dye (molecular weight: 668.15, 576 nm for excitation/601 nm for fluorescence). ALDOB, VAV2, D9S747, D9S303, and D9S304 were used as gene polymorphism sites from which loss of alleles was detected. After amplification reaction, the standard nucleic acid-derived amplified fragments and the measurement nucleic acid-derived amplification fragments were mixed in the same way as in Example 1.

TABLE 4

| | | | | |
|---|---|---|---|---|
| Gene | | Direc- | | |
| name | Label | tion | 5′-Sequence-3′ | SEQ ID NO |

Primers for standard nucleic acid amplification

| | | | | |
|---|---|---|---|---|
| ALD | FAM | Forward | GGG CTT GAC TTT | SEQ ID NO: 9 |
| | | | CCA ACA CG | |
| | None | Reverse | TCT AGC CTC AAT | SEQ ID NO: 10 |
| | | | CCT CAT AC | |
| VAV2 | None | Forward | GTG TCT GCA CTG | SEQ ID NO: 11 |
| | | | GCC ACA CT | |
| | FAM | Reverse | TCC AAA GGA CCT | SEQ ID NO: 12 |
| | | | TCT CCA AA | |
| 747 | None | Forward | GCC ATT ATT GAC | SEQ ID NO: 13 |
| | | | TCT GGA AAA GAC | |
| | FAM | Reverse | CAG GCT CTC AAA | SEQ ID NO: 14 |
| | | | ATA TGA ACA AAA T | |
| 303 | FAM | Forward | CAA CAA AGC AAG | SEQ ID NO: 15 |
| | | | ATC CCT TC | |
| | None | Reverse | TAG GTA CTT GGA | SEQ ID NO: 16 |
| | | | AAC TCT TGG C | |
| 304 | FAM | Forward | GTG CAC CTC TAC | SEQ ID NO: 17 |
| | | | ACC CAG AC | |
| | None | Reverse | TGT GCC CAC ACA | SEQ ID NO: 18 |
| | | | CAT CTA TC | |

Primers for measurement nucleic acid
amplification

| | | | | |
|---|---|---|---|---|
| ALD | ROX | Forward | GGG CTT GAC TTT | SEQ ID NO: 19 |
| | | | CCA ACA CG | |
| | None | Reverse | TCT AGC CTC AAT | SEQ ID NO: 20 |
| | | | CCT CAT AC | |
| VAV2 | None | Forward | GTG TCT GCA CTG | SEQ ID NO: 21 |
| | | | GCC ACA CT | |
| | ROX | Reverse | TCC AAA GGA CCT | SEQ ID NO: 22 |
| | | | TCT CCA AA | |
| 747 | None | Forward | GCC ATT ATT GAC | SEQ ID NO: 23 |
| | | | TCT GGA AAA GAC | |
| | ROX | Reverse | CAG GCT CTC AAA | SEQ ID NO: 24 |
| | | | ATA TGA ACA AAA T | |
| 303 | ROX | Forward | CAA CAA AGC AAG | SEQ ID NO: 25 |
| | | | ATC CCT TC | |
| | None | Reverse | TAG GTA CTT GGA | SEQ ID NO: 26 |
| | | | AAC TCT TGG C | |
| 304 | ROX | Forward | GTG CAC CTC TAC | SEQ ID NO: 27 |
| | | | ACC CAG AC | |
| | None | Reverse | TGT GCC CAC ACA | SEQ ID NO: 28 |
| | | | CAT CTA TC | |

4. Blunt-End Treatment and SSCP Electrophoresis

Blunt-end treatment was performed in the same way as in Example 1 to prepare samples for analysis, which were then subjected to SSCP electrophoresis.

5. Detection Time Correction

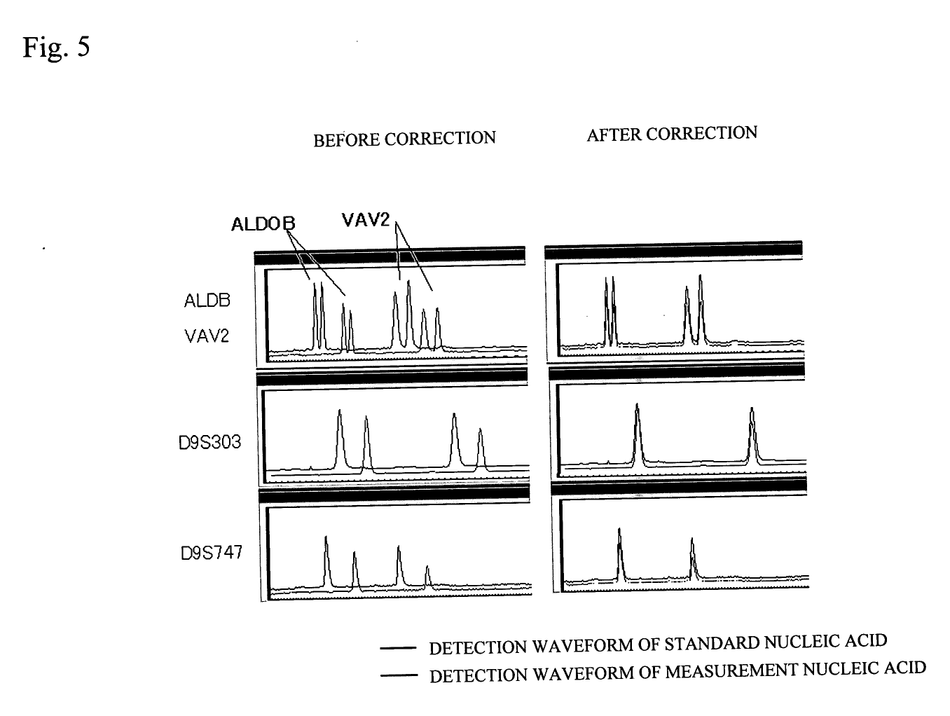

The standard nucleic acid labeled with the FAM fluorescent dye was detected with the peak of the standard nucleic acid amplified fragment having a fluorescence wavelength around 520 nm, while the measurement nucleic acid labeled with the ROX fluorescent dye was detected with the peak of the measurement nucleic acid amplified fragment having a fluorescence wavelength around 600 nm. In this detection, the ROX-labeled measurement nucleic acid had a molecular weight larger than that of the FAM-labeled standard nucleic acid and therefore resulted in a detection time approximately 3 seconds later, as shown in the left column of FIG. 5. Thus, the peak was allowed to move in parallel by image processing to correct the detection time difference by 3 seconds, as shown in FIG. 5. Then, the agreement of the standard nucleic acid-derived peak with the measurement nucleic acid-derived peak was confirmed in each gene region to make sure the absence of sample mix-up.

6. Measurement of Cancer Cell Ratio by LOH Analysis

When the peak intensity of either a paternal or maternal allele of a measurement nucleic acid extracted from the cancer tissue of a patient with cancer is decreased through the comparison of signals (peak intensities) between paternal and maternal alleles of a nucleic acid (i.e., standard nucleic acid) extracted from the blood of the same patient with cancer, it is defined as LOH. The ratio of a cancer-derived nucleic acid in the sample, that is, a cancer cell ratio, can be predicted according to the following formula (Genes, Chromosomes & Cancer, 15, p. 157-164, Sugano et al., (1996)):
Cancer cell ratio in measurement sample (%)=(NA1/NA2−TA1/TA2)×100/(NA1/NA2).

For a heterozygous human having A1 and A2 alleles, N denotes the peak intensity of a signal from the standard nucleic acid, that is, from the normal blood collected from the patient with cancer, and T denotes the peak intensity of a signal from the measurement nucleic acid, that is, from the cancer tissue of the same patient with cancer. A boundary value for LOH-positive/negative judgment (hereinafter, a cut-off value) was set to 10%. A cancer cell ratio (%) in the measurement sample was measured by using the calculation formula for calculating LOH (Table 5).

TABLE 5
LOH measurement result of each gene

| Gene name | ALDOB | VAV2 | D9S303 | D9S747 | Average |
|---|---|---|---|---|---|
| LOH value (%) | 15% | 16% | 15% | 14% | 15% |

As described above, the sample with a cancer cell ratio of approximately 15% can be measured quantitatively by labeling the nucleic acid fragments to prevent the peaks of the standard and measurement nucleic acids from overlapping and thereby detecting them in the same capillary.

As demonstrated by these results, the present invention has the following advantages:

1) nucleic acid fragments amplified form identical gene regions in standard and measurement nucleic acids can be detected simultaneously, with high quantitative properties maintained;

2) the correction of detection time is unnecessary after nucleic acid electrophoresis, leading to the cost reduction of internal control reagents for correction, the omission of an adding step, and the omission of correction procedures; and

3) since the difference between detection times derived from fluorescent dyes is always constant, waveform comparison can be recognized accurately by virtue of the absence of the difference between detection times difficult to correct, which is caused by difference in electrophoresis.

According to the present invention, nucleic acid fragments in identical gene regions of different origins can be detected simultaneously by one migration. The present invention can be applied to the quantitative analysis of polymorphism and mutation sites and is particularly useful in LOH analysis and cancer cell detection.

All publications, patents, and patent applications cited herein are incorporated herein by reference in their entirety.

[Free Text of Sequence Listing]

SEQ ID NOs: 1 to 28 Description of Artificial Sequences: Primers

What is claimed is:

1. A method of quantitative analysis of a gene region with plural possible sequences comprising: separately labeling a standard nucleic acid sample and a measurement nucleic acid sample with substances of different molecular weights; simultaneously separating and detecting nucleic acid fragments in the gene regions derived from the standard nucleic acid sample and from the measurement nucleic acid sample; and thereby quantitatively analyzing the gene region with plural possible sequences.

2. The method according to claim 1, wherein a means for separating and detecting nucleic acid fragments is electrophoresis.

3. The method according to claim 1, wherein a means for separating and detecting nucleic acid fragments is a single strand conformation polymorphism (SSCP) method.

4. The method according to claim 1, further comprising the step of separately amplifying the standard nucleic acid sample and the measurement nucleic acid sample with primers comprising the substances of different molecular weights.

5. The method according to claim 1, wherein the labeling substances of different molecular weights are fluorescent substances of different molecular weights.

6. The method according to claim 1, wherein the labeling substances of different molecular weights are any combination selected from FAM™ and ROX™, FAM™ and TAMRA™, fluorescein isothiocyanate (FITC) and Texas Red, and FITC and phycoerythrin.

7. The method according to claim 1, further comprising the step of correcting the difference between the detection times of the nucleic acid fragments by image processing.

8. The method according to claim 1, wherein the gene region with plural possible sequences is a polymorphism or mutation site.

9. A method of loss of heterozygosity (LOH) analysis, which utilizes a method according to claim 8.

10. A method of cancer cell detection, which utilizes a method according to claim 9.

11. A method of quantitative analysis of a gene region with plural possible sequences comprising:
1) separately labeling a standard nucleic acid sample and a measurement nucleic acid sample with substances of different molecular weights,
2) mixing the standard nucleic acid sample and the measurement nucleic acid sample,
3) separating and detecting nucleic acid fragments in mixed samples and analyzing their detection peaks, and
4) determining whether the detection peaks are derived from the standard nucleic acid sample or from the measurement nucleic acid sample.

12. The method according to claim 11, wherein the labeling is conducted by amplifying the standard nucleic acid sample and the measurement nucleic acid sample with primers comprising the substances of different molecular weights.

13. The method according to claim 11, wherein a means for separating and detecting nucleic acid fragments is electrophoresis.

14. The method according to claim 11, wherein the detection peaks are analyzed based on their detection times, peak intensities, and peak areas.

15. The method according to claim 11 further comprising comparing the detection times, peak areas, and peak intensities between the detection peaks derived from the standard nucleic acid sample and the measurement nucleic acid sample.
